(19) United States
(12) Patent Application Publication
(10) Pub. No.: US 2022/0118227 A1
(43) Pub. Date: Apr. 21, 2022

(54) TUBE SECUREMENT DEVICE WITH ADJUSTABLE SIZE

(71) Applicant: 3M INNOVATIVE PROPERTIES COMPANY, SAINT PAUL, MN (US)

(72) Inventors: DIANE J. ZIEMANN, FRIDLEY, MN (US); JEFFREY D. COTTON, SAINT PAUL, MN (US); AMANDA C. ENGLER, WOODBURY, MN (US); KIMBERLY A. SCHOMMER, HUDSON, WI (US); JAMES M. SIERACKI, PLYMOUTH, MN (US); MICHAEL J. TURNBULL, WOODBURY, MN (US); JENER DE OLIVEIRA, SUMARE-SP (BR); FERNANDA MARTINS, SAN PAULO (BR)

(73) Assignee: 3M INNOVATIVE PROPERTIES COMPANY, SAINT PAUL, MN (US)

(21) Appl. No.: 17/431,830

(22) PCT Filed: Mar. 6, 2020
(86) PCT No.: PCT/IB2020/051977

§ 371 Date: Aug. 18, 2021

Related U.S. Application Data
(60) Provisional application No. 62/817,707, filed on Mar. 13, 2019.

(51) Int. Cl. A61M 25/02

(52) U.S. Cl. CPC A61M 25/02; A61M 2025/0253; A61M 2025/0226; A61M 2025/022

(57) ABSTRACT

The disclosed tube securement device (100) is adjustable in size to fit varying sizes and shapes of a part of the body. The tube securement device (100) comprises a backing (110) forming a first end section (120), a second end section (130), and a midsection (140). A first adhesive (122) is on at least a portion of the first major surface (112) of the first end section (120). A second adhesive (132) is on at least a portion the second end section (130). A first perforation line (123) is through the first end section (120) to form a first removable portion (124). In use, the first removable portion (124) can be separated from the first end section (120).

### TECHNICAL FIELD

The present disclosure relates to a tube securement device with an adjustable size and methods of using a tube securement device with an adjustable size.

### BACKGROUND

At times, it may be necessary to insert tubes into a patient for different purposes, such as, feeding, air supply, and/or liquid removal. Such tubes generally need to be attached to the patient's skin in order to maintain the correction position of the tube.

Tubes inserted through the nose are referred to as a nasogastric (NG) tube and can be used for various applications, including feeding, drug administration and/or stomach drainage. Tubes inserted through the mouth are referred to an endotracheal tubes (ET) tubes. Tubes entering or exiting a body need to be securely attached to the patient's skin in order to maintain the correct position of the tube internally, such as inside the stomach, mouth, or airway. These tubed inside of a patient can be uncomfortable to the patient.

### SUMMARY

The disclosed tube securement device is adjustable in size to fit varying sizes and shapes of a body. Tape or other securement devices for securing tube within to skin will often adhere to the body, while adhesive strips wrap around the tubing and then connect with the tape or securement device on the body. Piecing together tape to fit on a patient can be time consuming. It is desirable to have a precut or preformed shape. However, individual's bodies vary in size and shape. For example, adults and children have very different size features, or one adult to another can have a different size nose, lips, cheeks. Therefore, it is desirable to have a securement device that can fit various sizes and shapes of a body.

The tube securement device comprises a backing having a first major surface and a second major surface, opposite the first major surface. The backing comprises a first end section, a second end section, and a midsection, separating the first end section from the second end section. The first end section, second end section, and midsection extend along a longitudinal direction. The first end section is wider than the midsection in a lateral direction that is perpendicular to the longitudinal direction. A first adhesive is on at least a portion of the first major surface of the first end section. A second adhesive is on at least a portion the second end section. A first perforation line is through the first end section to form a first removable portion. In use, the first removable portion can be separated from the first end section.

In one embodiment, the backing comprises a plurality of layers.

In one embodiment, the second end section is wider than the midsection in a lateral direction that is perpendicular to the longitudinal direction. In one embodiment, the first end section is wider than the midsection in a first lateral direction that is perpendicular to the longitudinal direction symmetrical and wider than the midsection in a second lateral direction that is opposite the first lateral direction. In one embodiment, the first end is symmetrical in the first lateral direction and second lateral direction. In one embodiment, the second end section is wider than the midsection in the lateral direction that is perpendicular to the longitudinal direction.

In one embodiment, the first adhesive and second adhesive are pressure sensitive adhesives. In one embodiment, wherein the device further comprises a third adhesive on at least a portion of the midsection. In one embodiment, the first major surface of the midsection is free of tacky adhesive and comprises one of: (i) a midsection adhesive composition detackified to form the first major surface that is free of tacky adhesive; (ii) a midsection adhesive composition covered to form the first major surface that is free of tacky adhesive, or (iii) no adhesive composition to form the first major surface that is free of tacky adhesive.

In one embodiment, the first perforation line begins at a first perimeter point of the first end section and extends to a second perimeter point of the first end section. In one embodiment, the first perforation line is a straight line and extends generally in the longitudinal direction. In one embodiment, the first perforation line is a curved line that concavely curves into the first end section. In one embodiment, the first perforation line is a curved line that convexly curves from the first end section. In one embodiment, the device further comprises a second perforation line through the first end section to form a second removable portion. In one embodiment, the second perforation line begins at a third perimeter point of the first end section and extends to a fourth perimeter point of the first end section. In one embodiment, the second perforation line is a straight line and extends generally in the longitudinal direction. In one embodiment, the second perforation line is a curved line that concavely curves into the first end section. In one embodiment, the second perforation line is a curved line that convexly curves from the first end section.

In one embodiment, the first removable portion and second removable portion are symmetrical about the first end section. In one embodiment, the first removable portion and second removable portion are concentrically arranged about the first end section.

In one embodiment, a system for securing a tube to skin comprises a tube securement device. The tube securement device comprises a backing having a first major surface and a second major surface, opposite the first major surface, wherein the backing comprises a first end section, a second end section, and a midsection, separating the first end section from the second end section. The first end section, second end section, and midsection extend along a longitudinal direction. The first end section is wider than the midsection in a lateral direction that is perpendicular to the longitudinal direction. A first adhesive on at least a portion of the first major surface of the first end section. A second adhesive on the second end section. A first perforation line through the first end section to form a first removable portion. The first adhesive of the first end section is secured to the skin. The second adhesive of the second end section is secured to the tube. In one embodiment, the first removable portion is removed from the tube securement device.

### BRIEF DESCRIPTION OF DRAWINGS

FIG. 1 is a plan view of one embodiment of a tube securement device, with first and second removable portions;

FIG. 2 is a plan view of the embodiment of the tube securement device of FIG. 1 with both first and second removable portions removed;

FIG. 3 is a side sectional view of the tube securement device of FIG. 1 through line 2-2;

FIG. 4 is a perspective view of the tube securement device of FIG. 2 applied to a nose and a tube;

FIG. 5 is a plan view of a second embodiment of a tube securement device, with first and second removable portions;

FIG. 6 is a plan view of a third embodiment of a tube securement device, with first and second removable portions;

FIG. 7 is a front view of a fourth embodiment of a tube securement device secured to a tube and to a person.

While the above-identified drawings and figures set forth embodiments of the invention, other embodiments are also contemplated, as noted in the discussion. In all cases, this disclosure presents the invention by way of representation and not limitation. It should be understood that numerous other modifications and embodiments can be devised by those skilled in the art, which fall within the scope and spirit of this invention. The figures may not be drawn to scale.

### DETAILED DESCRIPTION

The disclosed tube securement device is adjustable in size to fit varying sizes and shapes of a body. For example, the tube securement device may secure to a nose, lip, cheek, or other areas of skin. The tube securement device is formed from a backing and includes perforation lines through the backing to form removable portions.

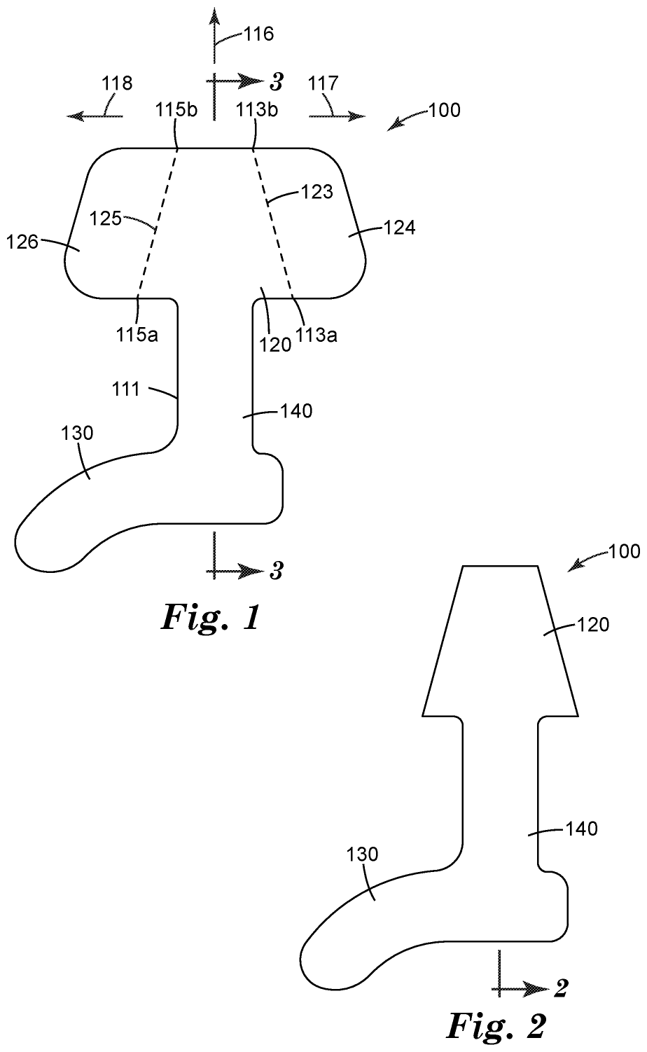

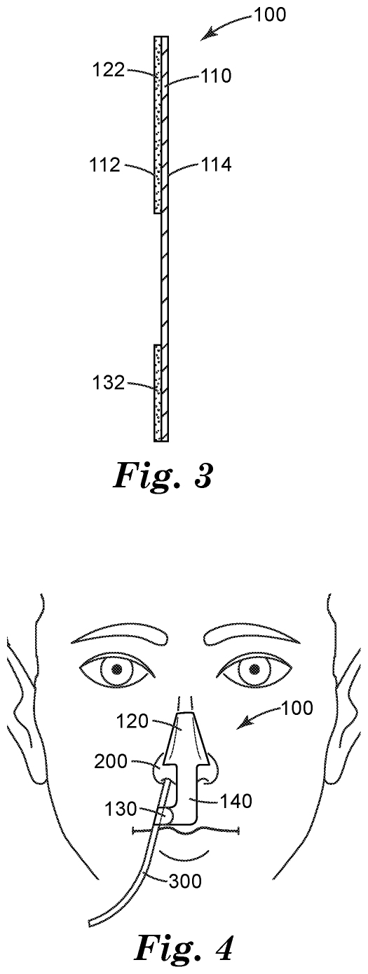

FIG. 1 is a plan view of one embodiment of a tube securement device 100 showing first removable portion 124 and second removable portion 126. FIG. 2 is a plan view of the tube securement device 100 of FIG. 1 with the first and second removable portions 124, 126 removed. FIG. 3 is a side sectional view of the tube securement device 100 of FIG. 1 through line 2-2. FIG. 4 is a front view of the tube securement device 100 of FIG. 2 secured to a tube 300 and to a nose 200.

The tube securement device 100 has a first major surface 112 and a second major surface 114, opposite the first major surface 112. A tube securement device 100 has a perimeter 111.

The tube securement device 100 comprises a backing 110. The backing 110 is made from a flexible material that will contour to the body 200 as well as the tube 300. Common materials for the backing 110 include paper, film, woven, knitted, nonwoven materials. The backing 110 can be a single layer or a multilayer construction. The backing 110 can be formed from materials that absorb or allow for transmission of moisture vapor generated from the body 200.

The tube securement device 100 comprises a first end section 120, a second end section 130, and a midsection 140, separating the first end section 120 from the second end section 130. Typically, the single, unitary backing material 110 material extends continuously from the first end section 120, the second end section 130, and the midsection 140, such as shown in the embodiment in FIGS. 1-4. It is understood, that the backing may comprises integrated sections that form the first end section 120, a second end section 130, and a midsection 140 such that the material forming each section is not identical to the other sections.

As shown in FIG. 1, the first end section 120, second end section 130, and midsection 140 extend along a longitudinal direction 116. In this embodiment, the first end section 120, second end section 130, and midsection 140 are linearly arranged along the longitudinal direction 116. It is understood that it is not necessary the first end section 120, second end section 130, and midsection 140 be linearly arranged, but could be offset or extend at a diagonal, for example.

The first end section 120 is wider than the midsection 140 in a lateral direction 117 that is perpendicular to the longitudinal direction 116. In the embodiment shown in FIGS. 1-4, the first end section 120 contacts the nose 200. Therefore, a wider portion of the first end section 120 allows for secure attachment to the nose 200, without the bulk of material overlying the tube 300 at the midsection 140.

In some embodiments, like shown in FIGS. 1, 5, 6, and 7 the second end section 130 is wider than the midsection 140 in a lateral direction 117 that is perpendicular to the longitudinal direction 116. When the first end section 120 is used to secure to the nose, the wider portion of the second end section 130 is secured to the tube 300 and allows for secure attachment to the tube 300 and possibly for wrapping around the tube 300 one or more times.

In some embodiments, like shown in FIGS. 1, 5, 6, and 7. Both the first end section 120 and the second end section 130 are wider than the midsection 140 in a lateral direction 117.

In some embodiment, like shown in FIGS. 1, 4, and 7, the first end section 120 is symmetrical in a first lateral direction 117 and second lateral direction 118 so that the first end section 120 can be place on the body 200. It is understood that this is not required and the first end section 120 can be of any variety of shapes and sizes to secure to a body. If securing to a nose, for example, the first end section 120 may be fitted to just a single side of the nose 200 and not centrally on the nose 200.

Similarly, the second end section 130 may extend in only in a first lateral direction 117, like shown in FIG. 1 or may extend symmetrically in a first lateral direction 117 and second lateral direction 118, like shown in the embodiment of FIG. 6.

Various shapes of the tube securement device suitable for the tube securement device 100 are shown in PCT publications: WO 2017/034907, WO 2017/034909, WO 2017/034911, WO 2017/034912, WO 2017/034913, and WO 2018/160649, the disclosures of which are herein incorporated by reference.

The first end section 120 is adjustable in size to accommodate for different sizes and shapes of the body 200. The first end section 120 includes a first perforation line 123 through the first end section 120 to form a first removable portion 124. To allow for the first removable portion 124 to separate from the first end section 120, the first perforation line 123 begins at a first perimeter point 113a, which is a point on the perimeter 111 of the first end section 120 and extends to a second perimeter point 113b, which is a point on the perimeter 111 of the first end section 120. As can be seen in FIG. 1 and FIG. 2, the first perforation line 123 allows the first removable portion 124 to be separated entirely from the first end section 120.

The first perforation line 123 can be in any number of shapes and lengths, so long as a portion of the first end section 120 can be removed. In the embodiment shown in FIG. 1, the first perforation line 123 is a straight line. This first perforation line 123 extends generally in the longitudinal direction but is slightly angled inward. This angled first perforation line 123 can provide for a secure fit when the first end section 120, with the first removable portion 124 removed, is applied to a face. The angled edge tends to follow the valley formed between the nose and cheek, as seen in FIG. 4.

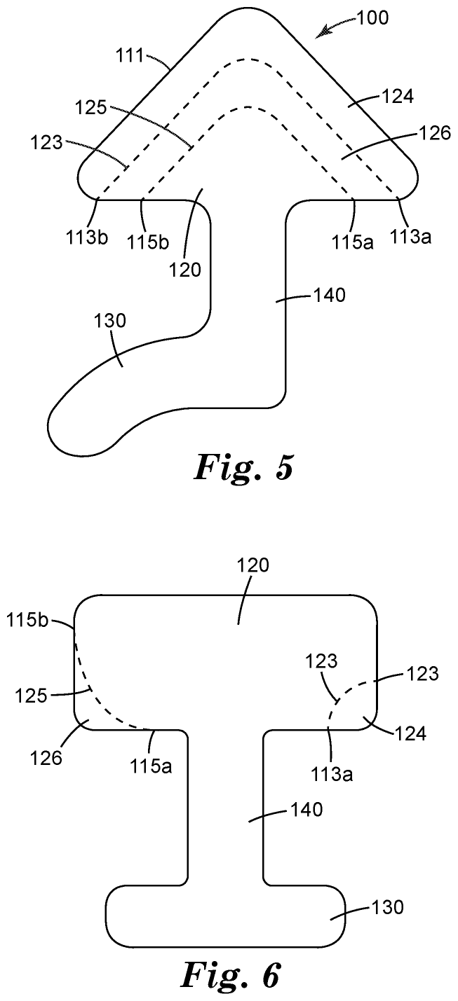

In other embodiments, the first perforation line is a curved line, like shown in FIGS. 5 and 6. In the curved line might concavely curve into the first end section 120, (see perforation line 123 in FIG. 6) or might convexly curve from the first end section 120, (see perforation line 125 in FIG. 6).

In one embodiment, the tube securement device 100 further comprises a second perforation line 125 through the first end section 120 to form a second removable portion 126. To allow for the second removable portion 126 to separate from the first end section 120, the second perforation line 125 begins at a third perimeter point 115a, which is a point on the perimeter 111 of the first end section 120 and extends to a fourth perimeter point 115b, which is a point on the perimeter 111 of the first end section 120. As can be seen in FIG. 1 and FIG. 2, the second perforation line 125 allows the second removable portion 126 to be separated entirely from the first end section 120. In other embodiments, further perforation lines may be included to provide for more adjustability of the first end section 120.

In the embodiment shown in FIG. 1, the second perforation line 125 is a straight line. This second perforation line 125 extends generally in the longitudinal direction but is slightly angled inward. This angled second perforation line 125 can provide for a secure fit when the first end section 120, with the second removable portion 126 removed, is applied to a face. The angled edge tends to follow the valley formed between the nose and cheek, as seen in FIG. 4. Similar to the first perforation line 123, the second perforation line can be of various sizes, shapes, lengths, curved or straight.

In the embodiment shown in FIG. 1-4, the first removable portion 124 and second removable portion 126 are symmetrically arranged about the first end section 120. Also, first removable portion 124 and second removable portion 126 are at opposite end sections of the first end section 120. Therefore, removal of one of the first or second removable portion 124, 126 could result in an asymmetrical first end section 120. This may be desirable to provide less of the tube securement device 100 on one side of the nose compared to the other.

FIGS. 5, 6, and 7 are alternative embodiment of a tube securement devices 100, using similar reference numbers to similar features as described above for FIGS. 1-4.

As shown in FIG. 5, the first end section 120 of the tube securement device 100 includes a first perforation line 123 through the first end section 120 to form a first removable portion 124. To allow for the first removable portion 124 to separate from the first end section 120, the first perforation line 123 begins at a first perimeter point 113a and extends to a second perimeter point 113b. The tube securement device 100 has an optional second perforation line 125 through the first end section 120 to form a second removable portion 126. To allow for the second removable portion 126 to separate from the first end section 120, the second perforation line 125 begins at a third perimeter point 115a and extends to a fourth perimeter point 115b. The first and second perforation lines 123, 125 is a curved line that is recessed from, but generally follows the shape of the perimeter 111 of the first end section 120.

In the embodiment shown in FIG. 5, the first removable portion 124 and second removable portion 126 are symmetrically arranged about the first end section 120. Also, first removable portion 124 and second removable portion 126 are concentrically arranged on the first end section 120. Removal of one of the first or second removable portion 124, 126 could result in a symmetrical first end section 120.

As shown in FIG. 6, the first end section 120 of the tube securement device 100 includes a first perforation line 123 through the first end section 120 to form a first removable portion 124. To allow for the first removable portion 124 to separate from the first end section 120, the first perforation line 123 begins at a first perimeter point 113a and extends to a second perimeter point 113b. The tube securement device 100 has an optional second perforation line 125 through the first end section 120 to form a second removable portion 126. To allow for the second removable portion 126 to separate from the first end section 120, the second perforation line 125 begins at a third perimeter point 115a and extends to a fourth perimeter point 115b.

In the embodiment shown in FIG. 6, the first removable portion 124 and second removable portion 126 are asymmetrically arranged about the first end section 120. The first perforation line 123 is a curved line concavely curves into the first end section 120. The second perforation line 125 is a curved line that convexly curves from the first end section 120.

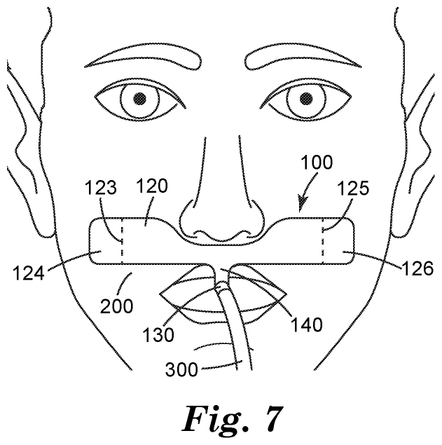

FIG. 7 is an embodiment of the tube securement device 100 that secured to a lip 200 and to a tube 300. In this embodiment the tube 200 is an endotracheal tube. Reference numbers in FIG. 7 identify the corresponding features as shown in FIG. 1. In this embodiment in FIG. 6, the tube securement device 100 include a first end section 120 with first adhesive (not apparent in this figure) secured to the body 200 at the upper lip and second end section 130 with second adhesive (not apparent in this figure) secured to the tube 300. The first end section 120 include first perforation line 123 and second perforation line 125. First removable portion 124 and second removable portion 126 remain connected to the first end section 120. The first end section 120 includes a recess for improving the fit in the narrow section between the nose and lip, so that larger sections of the first end section 120 can provide more contact area at the face. To use the tube securement device 100, the first end section 120 is secured to the body, such as the upper lip and cheeks, and the second end section 130 is secured to the tube 300.

Various shapes, sizes, and configurations of the perforation lines can be used interchangeable in the first end section to provide removable portions to the first end section. Removal of portions of the first end section provide for a more customized fit of the tube securement device.

The first major surface 112 of the first end section 120 contains an adhesive on the tube securement device 100. The second end section 130 contains an adhesive, which can be on either the first or second major surface 112, 114. Optionally the midsection may contain an adhesive. The adhesive is any suitable adhesive that will secure on contact to skin or secure to a tube. Such an adhesive may be referred to as having tack. For example, pressure sensitive adhesives, hydrogels, or hydrocolloids can be used.

A first adhesive 122 is on at least a portion of the first major surface 112 of the first end section 120. A second adhesive 132 is on at least a portion of the first major surface 112, second major surface 114, or both, of the second end section 130. FIG. 3 shows the first adhesive 122 and second adhesive 132 are both on the first major surface 112. As can be seen in FIG. 4, the first adhesive 122 at the first end section 120 secures to the nose 200, and the second adhesive 132 at the second end section 130 secures to the tube 300.

The adhesives can include any adhesive that provides acceptable adhesion to skin and is acceptable for use on skin (e.g., the adhesive should preferably be non-irritating and non-sensitizing). Suitable adhesives are pressure-sensitive and in certain embodiments have a relatively high moisture vapor transmission rate to allow for moisture evaporation. Suitable adhesives include those based on acrylates, urethane, hydrogels, hydrocolloids, block copolymers, silicones, rubber-based adhesives (including natural rubber, polyisoprene, polyisobutylene, butyl rubber etc.) as well as combinations of these adhesives. The adhesive component may contain tackifiers, plasticizers, rheology modifiers, absorbent particles or fibers as well as active components including for example an antimicrobial agent.

The adhesive used for the first adhesive 122, second adhesive 132, or third adhesive 142 may be compositionally the same adhesive. For example, all may be acrylate pressure sensitive adhesives or all may be silicone pressure sensitive adhesives. In other embodiments, the first adhesive 122, second adhesive 132, and third adhesive 142 are different from one another. It may be desirable to use one adhesive that is well suited for securing to skin, while a different adhesive is used that is suited for securing to tubing.

In some embodiments, the first major surface 112 of the midsection 140 is free of tacky adhesive. This means that the first major surface 112 of the midsection 140 will not secure on contact with skin or tubing. There are various ways of providing the first major surface 112 of the midsection 140 that is free of tacky adhesive. In one embodiment, such as shown in FIG. 3, at the midsection 140 no adhesive composition is applied at the first major surface 112 of the midsection and therefore the first major surface 112 is free of tacky adhesive.

In another embodiment, if the midsection 140 is covered with an adhesive composition, then the adhesive could be detackified so that the first major surface 112 is free of tacky adhesive. Adhesives can be detackified by using commonly known techniques, such as, for example, radiation curing or applying coatings.

In another example, if the midsection 140 is covered with an adhesive composition, then the adhesive composition could be covered to form the first major surface 112 that is free of tacky adhesive. The covering could be a film, paper, or other non-tacky material. Regardless of how it is formed, the midsection includes a first major surface 112 that is free of tacky adhesive.

To use the tube securement device 100, the first adhesive 122 at the first end section 120 may be applied to a nose 200 to secure the first end section 120 to the nose (see FIG. 4). To improve fit, the first removable portion 124 and/or second removable portion 126 (if included) can be removed before applying the first end section 120 to the nose 200 or after applying the first end section 120 to the nose 200.

The tube securement devices 100 disclosed herein can be manufactured using commonly used film converting, coating, cutting, and packaging techniques. For example, in one embodiment, the backing 110 is coated with the first adhesive 122, second adhesive 123, and/or third adhesive 142. Then, the coated backing is cut to the shape of the tube securement device 100. A release liner may be applied to entire first major surface 112 of the tube securement device.

Although specific embodiments have been shown and described herein, it is understood that these embodiments are merely illustrative of the many possible specific arrangements that can be devised in application of the principles of the invention. Numerous and varied other arrangements can be devised in accordance with these principles by those of skill in the art without departing from the spirit and scope of the invention. The scope of the present invention should not be limited to the structures described in this application, but only by the structures described by the language of the claims and the equivalents of those structures.

1-24. (canceled)

25. A tube securement device comprising:
a backing having a first major surface and a second major surface, opposite the first major surface, wherein the backing comprises a first end section, a second end section, and a midsection, separating the first end section from the second end section;
wherein the first end section, second end section, and midsection extend along a longitudinal direction;
wherein the first end section is wider than the midsection in a lateral direction that is perpendicular to the longitudinal direction;
a first adhesive on at least a portion of the first major surface of the first end section;
a second adhesive on at least a portion the second end section;
a first perforation line through the backing of the first end section to form a first removable portion.

26. The tube securement device of claim 25, wherein the second end section is wider than the midsection in a lateral direction that is perpendicular to the longitudinal direction.

27. The tube securement device of claim 25, wherein the first end is symmetrical about the longitudinal direction.

28. The tube securement device of claim 25, further comprising a third adhesive on at least a portion of the midsection.

29. The tube securement device of claim 25, wherein the first perforation line begins at a first perimeter point of the first end section and extends to a second perimeter point of the first end section.

30. The tube securement device of claim 25, wherein the first perforation line is one of (i) a straight line and extends generally in the longitudinal direction, (ii) a curved line that concavely curves into the first end section, or (iii) a curved line that convexly curves from the first end section.

31. The tube securement device of claim 25, further comprising a second perforation line through the first end section to form a second removable portion.

32. The tube securement device of claim 31, wherein the second perforation line begins at a third perimeter point of the first end section and extends to a fourth perimeter point of the first end section.

33. The tube securement device of claim 31, wherein the second perforation line is one of (i) a straight line and extends generally in the longitudinal direction, (ii) a curved line that concavely curves into the first end section, or (iii) a curved line that convexly curves from the first end section.

34. The tube securement device of claim 31, wherein the first removable portion and second removable portion are symmetrical about the first end section.

35. A tube securement device comprising:
a backing having a first major surface and a second major surface, opposite the first major surface, wherein the backing comprises a first end section, a second end section, and a midsection, separating the first end section from the second end section;
wherein the first end section, second end section, and midsection extend along a longitudinal direction;
wherein the first end section is wider than the midsection in a lateral direction that is perpendicular to the longitudinal direction;
a first adhesive on at least a portion of the first major surface of the first end section;
a second adhesive on at least a portion the second end section;
a first perforation line through the backing of the first end section to form a first removable portion;
a second perforation line through the backing of the first end section to form a second removable portion.

36. The tube securement device of claim 35, wherein the second perforation line is one of (i) a straight line and extends generally in the longitudinal direction, (ii) a curved line that concavely curves into the first end section, or (iii) a curved line that convexly curves from the first end section.

37. The tube securement device of claim 35, wherein the first removable portion and second removable portion are symmetrical about the first end section.

38. A system for securing a tube to skin comprising:
a tube securement device comprising:
a backing having a first major surface and a second major surface, opposite the first major surface, wherein the backing comprises a first end section, a second end section, and a midsection, separating the first end section from the second end section;
wherein the first end section, second end section, and midsection extend along a longitudinal direction;
wherein the first end section is wider than the midsection in a lateral direction that is perpendicular to the longitudinal direction;
a first adhesive on at least a portion of the first major surface of the first end section;
a second adhesive on the second end section;
a first perforation line through the first end section to form a first removable portion;
wherein, in use, the first adhesive of the first end section is secured to skin;
wherein, in use, the second adhesive of the second end section is secured to the tube.

39. The system for securing a tube of claim 18, wherein the first removable portion is removed from the tube securement device.

40. The system for securing a tube of claim 38, further comprising a second perforation line through the first end section to form a second removable portion.

41. The system for securing a tube of claim 38, wherein the second removable portion is removed from the tube securement device.
